(19) United States
(12) Patent Application Publication
(10) Pub. No.: US 2023/0102483 A1
(43) Pub. Date: Mar. 30, 2023

(54) [6R]-MTHF IN 5-FU BASED CHEMOTHERAPY OF LEFT-SIDED COLORECTAL CANCER

(71) Applicant: Isofol Medical AB, Goteborg (SE)

(72) Inventors: Roger Ingvar Tell, Saltsjo-Boo (SE); Lisa Katarina Skintemo, Lerum (SE); Per Emil Jonathan Holmen, Goteborg (SE); Per Lennart Lindberg, Goteborg (SE)

(21) Appl. No.: 17/488,120

(22) Filed: Sep. 28, 2021

Related U.S. Application Data
(63) Continuation of application No. PCT/EP2021/076513, filed on Sep. 27, 2021.

(51) Int. Cl. A61K 31/555; A61K 31/4745; A61K 9/00; A61K 31/513; A61K 31/519; C07K 16/22; A61K 47/12; A61K 9/08; A61P 35/00

(52) U.S. Cl. CPC A61K 31/555; A61K 31/4745; A61K 9/0019; A61K 31/513; A61K 31/519; C07K 16/22; A61K 47/12; A61K 9/08; A61P 35/00

(57) ABSTRACT

The present invention relates to the treatment of left-sided and rectal colorectal cancer in human populations having a high frequency of poor prognostic factors, including KRAS-mutated and wild-type (wt) colon cancer tumors, which involves administering multiple boluses of [6R]-5,10-methylenetetrahydrofolate ([6R]-MTHF) in connection with 5-fluorouracil (5-FU) based chemotherapy.

The instant application claims the benefit of priority under 35 U.S.C. § 119 to International Application No. PCT/EP2021/076513, filed Sep. 27, 2021, which is incorporated herein by reference in its entirety.

### FIELD OF THE INVENTION

The present invention relates to the treatment of solid tumors in humans with left-sided colorectal cancer or metastatic colorectal cancer, which comprises the administration of [6R]-5,10-methylene tetrahydrofolate (6R-MTHF) in 5-fluorouracil (5-FU) based chemotherapy, in combination with oxaliplatin or irinotecan.

### BACKGROUND OF THE INVENTION

Colorectal cancer (CRC) is one of the leading causes of mortality and morbidity in the world. With approximately 1,849,518 new cases estimated and 880,792 deaths per year (Caputo 2019), it also represents the third most common cancer worldwide and the second cause of cancer-related mortality, after lung cancer. In terms of geographical distribution, CRC incidence and prevalence have risen in industrialized countries (Bray 2018). Colorectal cancer affects approximately 135.439 estimated new patients in the United States per year. Of these cases, 39.910 per year (30%) are due to rectal cancer (Recio-Boiles 2020). However, in recent years the incidence and mortality rates of CRC have grown higher in Eastern Europe, Latin America, and Asia than other countries.

While the 5-year survival rate is 90% for early-stage CRC patients with localized disease, it is 70% for intermediate (regional invasive tumors) and 10% for advanced-stage patients with distant metastasis. Several factors including age, diet, hereditary polyposis syndrome and inflammatory bowel disease are associated with the development of CRC (Brenner 2014). However, CRC is not a single type of tumor; its pathogenesis depends on the anatomical location of the tumor and differs between right side and left side of the colon. Right-sided CRC (RCRC) tumors arise from the ascending colon, and proximal two thirds of the transverse colon whereas the left-sided CRC (LCRC) tumors arise from the descending and sigmoid colon, and distal one third of the transverse colon.

The importance of CRC sidedness in terms of treatment outcome was first addressed by Dr Alan Venook in 2016 (Venook 2016). For patients with metastatic colorectal cancer, he found that the sidedness of the primary tumor within the colon appears to affect both survival and the effectiveness of the commonly used biological agents Avastin (bevacizumab) and Erbitux (cetuximab), which are designed to interfere with the formation of blood vessels that feed a tumor, and with growth factor receptor signaling. Patients with left-sided disease enjoyed a median overall survival of 33 months compared with 19.4 months in right-sided disease. A further comparison of Avastin and Erbitux showed that Erbitux might be harmful to patients with right-sided tumors.

Tumors in the proximal colon (right-sided CRC, RCRC) and distal colon (left-sided CRC, LCRC) exhibit different molecular characteristics and histology. In fact, the two sides of the colon emerge from different parts of embryo. Some cells become the right colon, while others become the left colon. The right colon comes from the mid gut while the left colon comes from the hind gut, and they have different blood supplies. The cells on the left and right sides function slightly differently, and therefore, while they possess the same genes, they may not all be turned on to express the same set of proteins.

In the RCRC tumors, mutations in the DNA mismatch repair pathway are commonly observed; and these tumors generally have a flat histology. In the LCRC tumors, chromosomal instability pathway-related mutations, such as KRAS, APC, PIK3CA, p53 mutations are observed and these tumors demonstrate polypoid-like morphology. Therapy responses are totally different between these tumor entities. LCRC patients benefit more from adjuvant chemotherapies such as 5-fluorouracil (5-FU)-based regimes, and targeted therapies such as anti-epidermal growth factor receptor (EGFR) therapy, and have a better prognosis. RCRC patients do not respond well to conventional chemotherapies, but demonstrate more promising results with immunotherapies because these tumors have high antigenic load (Baran 2018).

Several publications highlight the differences between gene expressions in right- and left-sided CRCs. One example, which may explain the activity of epidermal growth factor receptor (EGFR) monoclonal antibodies (“mabs”), is that LCRC tumors overexpress genes involved in the EGFR pathway including the ligands for the EGFR receptor, epiregulin and amphiregulin. On the contrary, the higher methylation status of RCRC cancers results in these same genes being silenced (Burge 2019). Thus, anti-EGFR therapy benefits primarily those patients with left-sided or distal tumors (Hanna 2020).

Thus, CRC is no longer considered a single disease, rather it acts as two different diseases in the same organ (Baran 2018, Ross 2018). “Segregating patients according to RCRC v LCRC is a useful and pragmatic approach to guide decision-making regarding biological agents and should be adopted by oncologists in clinical practice” (Ross 2018).

On the basis of their molecular and clinical differences, left-sided and right-sided colorectal tumors are therefore now recognized as unique cancers that respond to different therapeutic strategies (Hanna 2020). See also Burge 2020 and FIG. 1 herein.

Trends in the incidence and mortality of colorectal cancer have however dramatically changed over the past decades. While the incidence and mortality among older (+50) adults has declined, the incidence of early-onset CRC (diagnosed before age 50 years) has nearly doubled since the 1990s. Early-onset CRC is now the second most common cancer diagnosis and third leading cause of cancer death in young adults.

A recently reported population-based study (Sanford 2020) conducted in the period 2000-2016 relates to 650,382 persons initially diagnosed with “generic” CRC, representing 66,186 (10.2% of total) and 584,196 (89.8% of total) incident cases of early-onset and older-onset CRC, respectively.

In this study, all-cause mortality generally increased with age. However, among those diagnosed before age 50 years, mortality was highest in the youngest age group (age 18-29 years), primarily driven by tumors of the left colon. Thus, the 5-year risk of death among 18-29 year olds with left-sided colon cancers exceeded that of nearly all other age groups. Younger adults were also more likely to present with left colon and rectal cancers and to be diagnosed with metastatic disease. Other characteristics of the study population were as follows:

A higher proportion of early-onset CRC occurred in men, racial/ethnic minorities and the left colon and rectum. Specifically, 30.0% of younger adults were diagnosed with rectal tumors compared with 20.5% of older adults, and there were notable differences in the proportion of tumors in the cecum (8.9 vs 16.2%), ascending colon (7.4 vs 13.7%) and sigmoid colon (21.5 vs 19.3%), by age (p<0.01). Younger adults also more often presented with metastatic disease (26.4 vs 20.6%, p<0.01).

Among those patients with right-sided colon cancer (n=262,713), there were marked differences in all-cause mortality by age at diagnosis. Mortality followed a linear trend (FIG. 2A). Compared with age 50-59 years, mortality was lowest at age 18-29 years and highest at age ≥70 years. Five-year risk of death similarly ranged from 0.27 to 0.57 for the youngest and oldest age groups, respectively.

In sharp contrast to right-sided colon cancer, age-related differences in mortality for left-sided colon cancer (n=169,830) followed a ‘J-shaped’ trend (FIG. 2B). Specifically, being diagnosed at age 18-29 years was associated with higher all-cause mortality when compared with 50-59 year olds. The 5-year risk of death for 18-29 year olds with left-sided colon cancer was higher than nearly every age group, except those ≥70 years, corresponding to risk differences ranging from 6 to 12%.

Mortality and 5-year risk of death for 30-39 and 40-49 year olds was similar to 50-59 year olds. Finally, for those with rectal cancer (n=187,826), the 5-year risk of death was highest for the oldest age groups but similar for all age groups less than 50 years. Compared with 50-59 year olds, mortality was slightly elevated among 18-29 year olds, although this was not statistically significant (FIG. 2C) (Sanford 2020).

Sanford further found that 71% of young adults in the study were diagnosed with left-sided colon or rectal cancers compared with 58% of older adults, and this difference contributed to higher risks of all-cause mortality, even for the youngest age group. Among those diagnosed between ages 18-29 and 30-39, mortality was actually greatest for left-sided colon cancers, even after adjusting for stage at diagnosis. Further, for the 18-29 years olds, mortality associated with left-sided colon cancer was higher than for the 50-59 years old and no better for 30-39 and 40-49 years olds.

This was surprising for several reasons: 1) younger adults are generally healthier and considered able to receive more aggressive therapies both at initial diagnosis and recurrence; 2) younger patients with tumors in the adjacent right colon fared best. In contrast, other studies generally report higher mortality among patients diagnosed with right-sided colon cancers, but these studies comprise predominantly older adults. Sanford's study therefore shows that not only are left-sided colon cancers enriched in younger adults, but they may also be disproportionately fatal. Similar findings were made by Mauri et al. who systematically retrieved 37 articles describing the prognosis of early-onset CRC compared with older patients and also found that the vast majority of early-onset CRC cases were left-sided (FIG. 3) and that the trends in CRC development are markedly different for the <50 and ≥50 years old (FIG. 4). Further, Mauri noted that since 1994, CRC incidence in individuals younger than 50 years has been increasing by 2% per year. Among the early-onset CRC patients, approximately 30% are affected by tumors harboring mutations causing hereditary cancer predisposing syndromes, and 20% have familial CRC. But notably, the remaining 50% of the early-onset CRC patients have neither hereditary syndromes nor familial CRC. There is presently no “good explanation” for this group.

By 2030 in the USA, 10% of all colon and 22% of all rectal cancers are now expected to be diagnosed in patients <50 years old, which is alarming compared with 4% and 9% for colon and rectal cancer, respectively, 10 years ago. Similarly, recent early-onset CRC data from Europe indicate an annual 1.5% increase in rectal cancer incidence between 1990 and 2008, and an annual 7.4% increase in colon cancer incidence between 2008 and 2016. These data indicate that early-onset-CRC is an increasing public health issue in the western world.

A key question in understanding the factors responsible for the increasing incidence of early-onset CRC is whether this is the same disease as late-onset CRC, or if early-onset is caused by unique underlying biological mechanisms that are impacted by different risk factors. Most early-onset CRC is found in the rectum and distal colon, and the tumors are microsatellite stable, chromosomal instable and have significantly fewer BRAF mutations than late-onset CRC, but there is so far (in 2021) no compelling evidence for distinguishing early-from late-onset CRC, and international CRC clinical guidelines accordingly do not use age as a determinant of early-onset CRC therapy. However, early-onset CRC patients tend to be diagnosed at later stages and receive more aggressive treatment, which may improve outcomes relative to older patients, but can also have a significant impact on quality of life. Data comparing survival in younger and older patients is inconsistent, although worse survival is observed for patients younger than 35 years old, perhaps attributable to a delay in diagnosis.

Therapeutic options for early- and late-onset CRC patients are thus currently the same according to the major oncology societies worldwide (Mauri 2019). Already 20 years ago Sargent et al. conducted a meta-analysis of seven trials regarding the use of 5-fluorouracil (5-FU)+leucovorin/folinic acid (LV) in the adjuvant setting. The analysis revealed no significant difference in efficacy or toxicity among patients aged 70 years or younger compared with patients older than 70 years (Sargent 2001). The pooled analysis, based on the intention to treat, of individual patient data from seven phase 3 randomized trials (involving 3351 patients) in which the effects of postoperative fluorouracil plus leucovorin (five trials) or fluorouracil plus levamisole (two trials) were compared with the effects of surgery alone in patients with stage II or III colon cancer. The patients were grouped into four age categories of equal size, and analyses were repeated with 10-year age ranges (< or =50, 51 to 60, 61 to 70, and >70 years), with the same conclusions. Adjuvant treatment had a significant positive effect on both overall survival and time to tumor recurrence (P<0.001 for each, with hazard ratios of death and recurrence of 0.76 [95 percent confidence interval, 0.68 to 0.85] and 0.68 [95 percent confidence interval, 0.60 to 0.76], respectively). The five-year overall survival was 71 percent for those who received adjuvant therapy, as compared with 64 percent for those untreated. No significant interaction was observed between age and the efficacy of treatment. The incidence of toxic effects was not increased among the elderly (age >70 years), except for leukopenia in one study.

The “EGFRmabs” cetuximab and panitumumab are currently recommended in first line treatment of LCRC tumors (for both early-onset and late onset CRC) and have been incorporated into international guidelines recommending their use, including in patients harboring RAS (wt), in conjunction with folate enhanced 5-FU treatment protocols together with either oxaliplatin or irinotecan (5-FU+leucovorin+oxaliplatin [FOLFOX] or 5-FU+leucovorin+irinotecan [FOLFIRI]). Specifically, the best survival outcomes for LCRC RAS (wt) patients are achieved when combination chemotherapy (incorporating 5-FU plus an EGFRmab such as cetuximab or panitumumab) is used, while reserving bevacizumab for second-line use (Burge 2019).

However, both cetuximab and panitumumab have severe side effects. The following specific side effects are commonly (i.e. occurring in 10-30% or more of all treated patients) associated with being treated with these compounds (source: http://chemocare.com/):

TABLE 1
Common side effects associated with cetuximab and panitumumab

| cetuximab | panitumumab |
|---|---|
| Skin changes such as acne-like | Anxiety. |
| rash, or dry, itchy or flaky skin. | Bloating or swelling of the face, arms, |
| Reduced levels of magnesium in | hands, lower legs, ankles, or feet. |
| the blood (hypomagnesemia). | Convulsions. |
| Diarrhoea. | Decreased urination. |
| Allergic reactions. | Deep cracks, grooves, or lines in the |
| Heart attack. | skin. |
| Abnormal hair growth. | Acneform dermatitis and itching |
| Swelling of skin around nails | Reduced levels of magnesium in the |
| Blistering of the skin or mucous | blood (hypomagnesemia). |
| membranes (such as the mouth) or | Difficulty with swallowing. |
| peeling of the skin. | Discoloration of the nails. |
| | Drowsiness. |

There thus remains a need for an improved folate-enhanced 5-FU treatment protocol for left-sided and rectal CRC tumours, including in patients determined by genotype testing to be BRAF or KRAS mutation-positive, both in general and in particular with a view to treating early-onset (18-49 years) CRC patients that typically present primary tumors located in the left colon or rectum, and frequently also have metastatic disease.

### Definitions

As used herein, the term Leucovorin® or folinic acid shall both mean 5-formyl tetrahydrofolic acid, i.e. the 5-formyl derivative of tetrahydrofolic acid. Folinic acid contains 2 asymmetric centers. Commercially available leucovorin (LV) is composed of a 1:1 mixture of the dextrorotary and levorotary diastereomers (d-leucovorin (d-LV, (6R,2'S)-configuration) and 1-leucovorin (1-LV, (6S,2'S)-configuration), resp.), and may also be referred to as (d,l-LV).

As used herein, the term Levoleucovorin shall refer to the commercially available product which contains only the pharmacologically active levo-isomer 1-LV (or LLV). In vitro, the levo form 1-LV has been shown to be rapidly converted to the biologically available methyl-tetrahydrofolate form while the dextro form d-LV (DLV) is slowly excreted by the kidneys. Leucovorin and levoleucovorin have however been shown to be pharmacokinetically identical and may be used interchangeably with limited differences in efficacy or side effects (Kovoor et al, Clin Colorectal Cancer 8 200-6 (2009).

As used herein, the terms MTHF or methylene THF shall both refer to 5,10-Methylene-5,6,7,8-tetrahydrofolate.

As used herein, the terms racemic methyleneTHF, CoFactor® or [6R,S]-5,10-methyleneTHF shall all refer to the 1:1 diastereomeric mixture [6R, S]-5,10-Methylene-5,6,7,8-tetrahydrofolate.

As used herein, the term [6R]-5,10-MTHF shall refer to the single diastereomer, [6R]-5,10-methylenetetrahydrofolate.

As used herein, the terms IV or i.v. shall both mean intravenous.

As used herein, the term DLT shall refer to dose-limiting toxicity. Dose Limiting Toxicity (DLT) is a medical occurrence that is assessed as at least possibly related to a pharmaceutical product (i.e. to one or more chemotherapeutic agents) and is severe enough to prevent further increase in dosage or strength of treatment agent, or to prevent continuation of treatment at any dosage level.

As used herein, the term ORR shall refer to the Objective Response Rate, ie. the proportion of patients with reduction in tumor burden of a predefined amount. This shall be calculated as follows: ORR=(Sum of partial responses plus complete responses) divided by total number of observations, as per RECIST 1.1 (a set of published rules that define when tumors in cancer patients progress during treatments, the responses being defined as:

Complete Response (CR):
Disappearance of all target lesions. Any pathological lymph nodes (whether target or non-target) must have reduction in short axis to <10 mm.

Partial Response (PR):
At least a 30% decrease in the sum of diameters of target lesions, taking as reference the baseline sum diameters.

Progressive Disease (PD):
At least a 20% increase in the sum of diameters of target lesions, taking as reference the smallest sum on study (this includes the baseline sum if that is the smallest on study).
In addition to the relative increase of 20%, the sum must also demonstrate an absolute increase of at least 5 mm. (Note: the appearance of one or more new lesions is also considered progression).

Stable Disease (SD):
Neither sufficient shrinkage to qualify for PR nor sufficient increase to qualify for PD, taking as reference the smallest sum diameters while on study.
(Eisenhauer E A, Therasse P, Bogaerts J, Schwartz L H, Sargent D, Ford R, et al. New response evaluation criteria in solid tumors: revised RECIST guideline (version 1.1). Eur. J Cancer. 2009 January; 45(2):228-47)

As used herein, the term dU shall refer to deoxyuridine.

As used herein, the term BSA refers to Body Surface Area

As used herein, the terms BRAF mutation-positive patients and KRAS mutation-positive patients shall refer to patients who by genotype testing have been found to harbor either BRAF- or KRAS mutated tumors and/or metastases.

As used herein, the term ctDNA genotype testing shall refer to genotype testing conducted by analyzing a blood or serum sample for cell-free tumor DNA.

As used herein, the term right-sided colorectal cancer shall refer to patient cases wherein the primary tumor of said patient has been determined to be located in the cecum, ascending colon, hepatic flexure and/or transverse colon. As used herein, the term left-sided colorectal cancer (LCRC) shall refer to patient cases wherein the primary tumor of said patient has been determined to be located in the distal one-third of the transverse colon, the descending colon and the sigmoid colon. LCRC may also include cases wherein the primary tumor of said patient is located in the rectum.

### SUMMARY OF INVENTION

Arfolitixorin (Modufolin®) is a new drug developed to increase the efficacy of the cytotoxic agent 5-fluorouracil (5-FU) and as a rescue drug after high-dose methotrexate treatment. The active ingredient in arfolitixorin (Modufolin®), [6R]-5,10-methylenetetrahydrofolate, abbreviated herein as [6R]-5,10-MTHF, is the key active metabolite of the widely used folate-based drugs leucovorin and levoleucovorin. Arfolitixorin (Modufolin®) is suitable for all patients irrespective of their capacity to activate folates since it does not require metabolic activation to exert its effect.

According to the present invention, it has surprisingly been found that by treating patients diagnosed with left-sided metastatic colorectal cancer according to chemotherapeutic protocols involving initial administration of 5-FU followed by administration of [6R]-5,10-MTHF, ORRs (objective response rates) of app. 70% can be achieved. It has further been found that patients diagnosed with either left-sided metastatic colorectal cancer or rectal cancer achieve ORRs of >60%.

Accordingly, in a first aspect of the invention, [6R]-5,10-methylenetetrahydrofolate ([6R]-5,10-MTHF) is provided for use in a human in the treatment of colorectal cancer tumors, which treatment comprises the following steps:
a) administering a continuous IV infusion containing 85 mg/m² (of BSA) ox-aliplatin, followed by
b) administering an IV bolus containing 400 mg/m² (of BSA) 5-fluorouracil, followed by
c) administering an IV bolus containing 60 mg/m² [6R]-5,10-methylenetetrahydrofolate, followed by
d) administering a continuous IV infusion containing 2400 mg/m² 5-fluorouracil over 46 hours±1 hour followed by
e) administering an IV bolus containing 60 mg/m² (of BSA) [6R]-5,10-methylenetetrahydrofolate,
   wherein the location of the primary tumor of said patient has been determined to be left-sided or rectal, and wherein all steps a)-e) are repeated every 2 weeks until termination of the treatment, and wherein all steps a)-e) are repeated every 2 weeks for at least 16 weeks, until termination of the treatment.

In a second aspect of the invention, [6R]-5,10-methylenetetrahydrofolate ([6R]-5,10-MTHF) is provided for use in a human in the treatment of solid colorectal cancer tumors, which treatment comprises the following steps
a) administering a continuous IV infusion containing 180 mg/m² (of BSA) irinotecan, followed by
b) administering an IV bolus containing 400 mg/m² (of BSA) 5-fluorouracil, followed by
c) administering an IV bolus containing 60 mg/m² [6R]-5,10-methylenetetrahydrofolate, followed by
d) administering a continuous IV infusion containing 2400 mg/m² 5-fluorouracil over 46 hours±1 hour followed by
e) administering an IV bolus containing 60 mg/m² (of BSA) [6R]-5,10-methylenetetrahydrofolate,
   wherein the location of the primary tumor of said patient has been determined to be left-sided or rectal, and wherein all steps a)-e) are repeated every 2 weeks until termination of the treatment, and wherein all steps a)-e) are repeated every 2 weeks for at least 16 weeks, until termination of the treatment.

The treatment based on the ARFOX or ARFIRI protocol may in principle be terminated “for any reason”, such as e.g. by a patient decision or a decision taken by the responsible medical person, i.a. due to disease progression or adverse events. Furthermore, the ARFOX or ARFIRI protocol may be interrupted by treatment holidays and the like. Finally the responsible medical person may decide on a fixed number of treatment cycles.

It has also surprisingly been discovered that administration of [6R]-MTHF and 5-FU according to the first or second aspect of the present invention over a treatment period of at least 16 weeks lead to a retardation or prevention of the progression of left-sided or rectal, colorectal cancer tumors in a human patient.

In a third aspect of the invention [6R]-5,10-methylene-tetrahydrofolate is therefore provided for use in the retardation or prevention of the progression in a human patient of left-sided or rectal colorectal cancer tumors, which comprises performing and repeating steps a) to e) according to the first or second aspect of the present invention, over a total treatment period of at least 16 weeks.

In a fourth aspect of the invention, there is provided a method for retardation or prevention of the progression in a human of left-sided or rectal colorectal cancer tumors, which method comprises performing and repeating steps a) to e) according to the first or second aspect of the present invention, over a total treatment period of at least 16 weeks.

### BRIEF DESCRIPTION OF FIGURES

FIG. 1 (adapted from Burge 2019): An overview of differences between LCRC and RCRC.

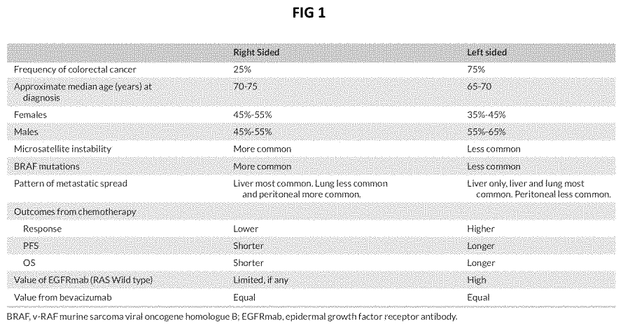

FIG. 2 (adapted from Sanford 2020): Association between age at diagnosis and all-cause mortality by tumour side. Adjusted hazard ratios (HR) demonstrating association between age at diagnosis and all-cause mortality for right colon (A), left colon (B), and rectal (C) cancer, SEER 18, 2000-2016. HR: Hazard ratio; SEER: Surveillance, Epidemiology and End Result.

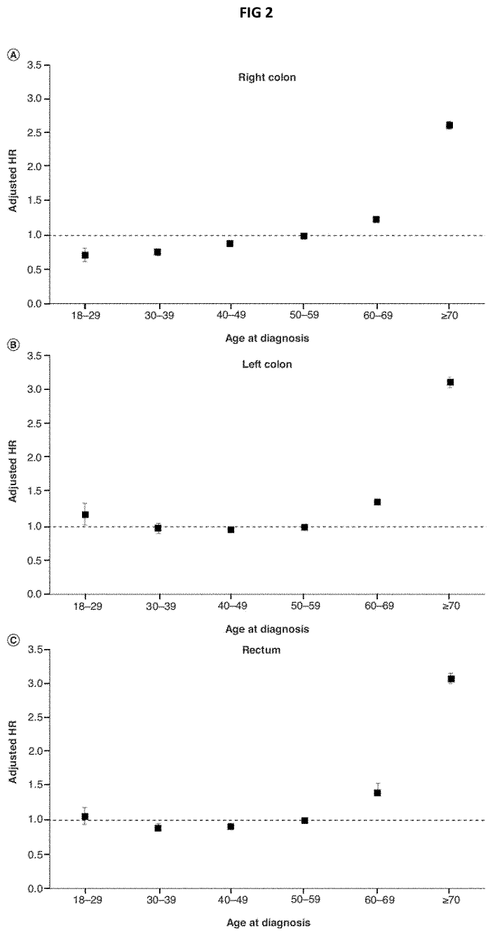

FIG. 3: (Table 2 adapted from Mauri 2019). As can be seen from the 3rd column in the table (column named “Primary tumor in left colon or rectum (%)”), the percentage of tumors found in either left colon or rectum in the particular reference is significantly above 50% for all the cited references. The lowest cited percentage is 62% (reference: Watson et al (2016)) and the highest is 91% (Yantiss et al. (2009)).

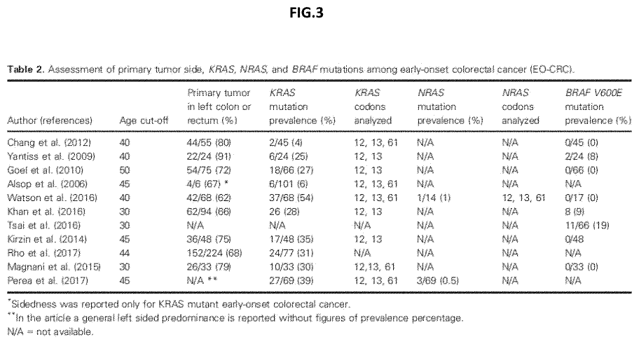

FIG. 4: (FIG. 2 adapted from Mauri 2019). Graphs report age-adjusted SEER (Surveillance, Epidemiology and End Results) incidence rates of colon (upper panels) and rectal (lower panels) cancer from 1975 to 2015 among individuals younger (left panels) and older (right panels) than 50 years. On the Y-axis is reported incidence rate per 100 000 and on the X-axis is reported the year of diagnosis. Data were plotted by accessing SEER web site at the weblink https://seer.cancer.gov/faststats/selections.php?series=cancer

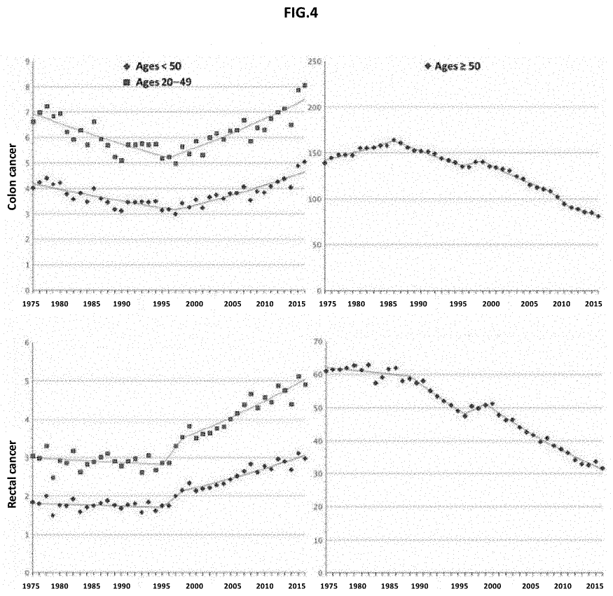

FIG. 5: An overview of treated patients in the follow-up study discussed herein.

### DETAILED DESCRIPTION OF THE INVENTION

Arfolitixorin has been in development for a number of years and has been studied in several clinical studies. During one of these studies (the Phase Ulla study ISO-CC-005) it was surprisingly discovered in December 2017 that administration of [6R]-MTHF and 5-FU according to a particular treatment regimen over a treatment period of at least 8 weeks lead to a prevention or retarding of the progression in a human of tumors. No statistically significant progression of said tumors was observed between 8 and 16 weeks after initiating treatment. These results are discussed i.a. in applicant's international patent application WO 2019/037899 published 28 Feb. 2019. The completion of the study was announced in January 2020. In total, 105 patients were included in the study.

Applicant completed the dose definition part of ISO-CC-005 in March 2018, which evaluated the safety and efficacy of arfolitixorin in patients with mCRC. Shortly after, applicant started two additional treatment groups in 2018 to generate more safety and efficacy data, i.e. the safety extension Cohort #18 (Treatment Arm #4) and Cohort #19 (Treatment Arm #6). The aim was to evaluate as many patients as possible from the additional treatment groups after a treatment period of 16 weeks or more.

On 30 Sep. 2020, applicant announced response assessment data from the two safety extension cohorts (31 patients) treated for 16 weeks or longer (press release: 55% Overall Response Rate on the safety extension cohorts of the ISO-CC-005 Phase I/IIa study|BioSpace). The data showed a best overall response rate (ORR) of 55%. These patients had been treated with the selected dose regimen of 120 mg/m2 arfolitixorin and 5-fluorouracil (5-FU) with either irinotecan or oxaliplatin (ARFIRI/ARFOX). Out of the 31 patients, 17 were treated with an ARFOX regimen.

A best ORR of 59% was observed in the ARFOX regimen group versus 50% in the ARFIRI regimen group, despite that 53% of the patients had a right-sided tumor location and 24% were carrying a BRAF mutation. As mentioned above, in the general CRC population and in historical control first line mCRC Phase III trials, a percentage of approximately 30-40% right-sided tumors are seen and around 10% of the patients carry a BRAF mutation. Both right-sided tumor location and BRAF mutations are historically known as poor prognostic factors and the best ORR in these patient populations in the first line mCRC setting treated with either FOLFOX or FOLFIRI historically generates best ORRs in the range of ˜40% and 15-20% respectively (see eg Loupakis 2018, Van Cutsem 2015 and Tveit 2012).

The average ORR based on pivotal Phase III trials considered in a recent meta-analysis/review indicates that FOLFOX regimens generates 45% best ORR and FOLFIRI regimens generates 40% best ORR in historical non-selected patient population (all-comer), first line mCRC populations (Giuliani 2018).

During a subsequent analysis of the follow-up study results, applicant has now discovered that some of the tested combinations have proven surprisingly effective against left-sided and rectal CRC, including in cases where the patients were determined by genotype testing to be KRAS- or BRAF-mutation positive. As mentioned hereinabove, these tumor types are particularly aggressive and difficult to treat. Left-side and rectal CRC cancers are also prominent amongst the early-onset (18-49 years old) cancer patients as discussed hereinabove. Applicant believes that even if the ISO-005 and its follow-up study described herein were not specifically designed to assess the efficacy of the ARFIRI/ARFOX protocols in this patient group, it is highly likely that the efficacy of the two protocols demonstrated herein will also positively impact on the treatment of early-onset (18-49 years) CRC patients.

The baseline CRC genotype and sidedness status for the 43 patients enrolled in the two additional treatment groups was collected and summarized as follows:

| Primary tumor location | KRAS status | BRAF status |
|---|---|---|
| Left sided 16 (37%) | Mutant: 17 (40%) | Mutant 9 (21%) |
| Right sided 19 (44%) | Wild type 22 (51%) | Wild type 25 (58%) |
| Rectum 8 (19%) | Unknown 4 (9%) | Unknown 9 (21%) |

Of the 43 patients enrolled in the two safety extension cohorts, 12 patients were either not evaluated with a CT-scan at 8 weeks or beyond 8 weeks of treatment. Of these 12 patients, 4 had Stable Disease (SD) and 3 had Partial Response (PR) already after 8 weeks.

Of the 31 patients actually evaluated at 16 weeks (or more), 13 (42%) had RCRC, 13 (42%) had LCRC, 5 (16%) had rectal CRC (RCA), 8 (26%) BRAF-mutated CRC and 11 (35%) KRAS-mutated(mt) LCRC, which corresponds roughly with the composition of the initial group of 43 patients enrolled in the two safety extension cohorts.

The objective response rates (ORRs) for left- and right-sided CRC and rectal cancer have historically been found to be quite different. Patients with LCRC, regardless of the type of treatment received, have superior ORRs (app. 54% vs 41%, see eg. Grassadonia 2019).

The preferred adjuvant chemotherapy for RCA is an oxaliplatin-containing regimen (FOLFOX, CAPDX) administered for two months, followed by a fluoropyrimidine-based CRT (chemo+radiation therapy) and completed by two months of additional chemotherapy. Alternative four months of chemotherapy, followed by two months of CRT, is acceptable. Fluoropyrimidine radiation sensitizer current regimens include weekly bolus 5-FU (the Mayo and Roswell Park regimen), short-term infusional FU (the de Gramont regimen), and single-agent capecitabine. At this point, neither irinotecan nor targeted-based therapies have an indication in the adjuvant therapy of RCA outside of investigational trials. (Recio-Boiles 2020).

Clinical studies citing substantiated ORRs specifically for rectal cancer (RCA) patients (rather than CRC in general) treated with 5-FU+leucovorin have not been found published. However, according to Cancer.net (Colorectal Cancer: Statistics|Cancer.Net), the overall 5-year survival rate for people with rectal cancer is 67%. If the cancer is diagnosed at a localized stage, the survival rate is 89%. If the cancer has spread to surrounding tissues or organs and/or the regional lymph nodes, the 5-year survival rate is 72%. If the cancer has spread to distant parts of the body, the 5-year survival rate is 16%. These figures are very similar to the statistics for colorectal cancer in general. For the purpose of further discussion herein, the ORRs for RCA patients when treated with an oxaliplatin/5-FU based regimen will be assumed on par with ORRs for LCRC patients.

Apart from the sidedness, the historical ORRs have also been found to be very different for patients with BRAF wild-type (wt) vs. mutant tumors. ORRs were thus found to be 15-20% in patients with BRAF-mutant tumors vs. 50% in those with BRAF (wt) tumors (Li 2020).

The ORRs have on the other hand in a number of studies been found to be quite similar (around 42%) for patients with KRAS (wt)-type vs. KRAS-mutant CRC. Thus, in a Chinese study comprising 141 patients with known KRAS status, 55 patients had KRAS mutation and 86 had KRAS wild-type. The group was treated according to different chemotherapeutic regimens, most including bevacizumab, and achieved ORR and DCR of 41.9% and 78.9% in patients with KRAS (wt), while the ORR and DCR were 38.7% and 77.9% in patients with KRAS mutation (Sun 2017). This similarity was confirmed in a Greek study with patients in first line chemotherapy with bevacizumab. The ORRs for FOLFOX/BEV was 48.3% (115 pts), FOLFIRI/BEV 47.7% (92 pts) and XELOX/BEV 45% (65 pts), regardless of KRAS status (Koumarianou 2018).

Several clinical studies have thus demonstrated the following approximate ORRs for LCRC with or without mutations:
ORR=54% for LCRC without mutations
ORR=20% for CRC with BRAF mutations
ORR=42% approximately identical for LCRC with KRAS mutations or KRAS wild type.

Returning now to the 31 patients from the follow-up study evaluated at 16 weeks (or more), and the “LCRC or RCA” group of 18 patients. Upon further analysis this group contained:
5 cases of LCRC or RCA with BRAF mutations (28%)
13 cases of LCRC or RCA with either BRAF wild type or unknown type (72%).

This group would be expected to have an overall best ORR reflecting the proportion of patients with LCRC or rectal (no mutations)+the proportion of patients with BRAF(mt) LCRC which can be calculated as follows:

Expected ORR_{LCRC+rectal group}=Prop_{BRAF(mt)}*ORR_{BRAF(mt)}+PrOP_{LCRC}*ORR_{LCRC}

Which gives, using the historical ORR's discussed above:

Expected ORR_{LCRC group}=28%*15-20%+72%*54%=app. 44%

However, when assessing the results for the 31-patient group after 16 weeks and beyond of treatment with 120 mg/m2 arfolitixorin+5-FU+irinotecan or oxaliplatin (ARFIRI/ARFOX), it was surprisingly found that patients in the 31-patient group diagnosed with LCRC or RCA had a best ORR of 61%. Without the rectal cases (i.e. only LCRC), the best ORR were 69%.

Of the 18 patients diagnosed with LCRC or RCA, 11 were determined to be either BRAF or KRAS-mutation positive by genotype testing. Thus, the ARFIRI/ARFOX treatment protocols have proven surprisingly effective in the treatment of patients with left-sided CRC and rectal cancer (RCA), including patients who by genotype testing have been determined to be BRAF- or KRAS mutation-positive.

Accordingly, in a first aspect of the invention, [6R]-5,10-methylenetetrahydrofolate ([6R]-5,10-MTHF) is provided for use in a human in the treatment of colorectal cancer tumors, which treatment comprises the following steps:
a) administering a continuous IV infusion containing 85 mg/m² (of BSA) oxaliplatin, followed by
b) administering an IV bolus containing 400 mg/m² (of BSA) 5-fluorouracil, followed by
c) administering an IV bolus containing 60 mg/m² [6R]-5,10-methylenetetrahydrofolate, followed by
d) administering a continuous IV infusion containing 2400 mg/m² 5-fluorouracil over 46 hours±1 hour followed by
e) administering an IV bolus containing 60 mg/m² (of BSA) [6R]-5,10-methylenetetrahydrofolate,
   wherein the location of the primary tumor of said patient has been determined to be left-sided or rectal, and wherein all steps a)-e) are repeated every 2 weeks until termination of the treatment, and wherein all steps a)-e) are repeated every 2 weeks for at least 16 weeks, until termination of the treatment.

In a second aspect of the invention, [6R]-5,10-methylenetetrahydrofolate ([6R]-5,10-MTHF) is provided for use in a human in the treatment of solid colorectal cancer tumors, which treatment comprises the following steps
a) administering a continuous IV infusion containing 180 mg/m² (of BSA) irinotecan, followed by
b) administering an IV bolus containing 400 mg/m² (of BSA) 5-fluorouracil, followed by
c) administering an IV bolus containing 60 mg/m² [6R]-5,10-methylenetetrahydrofolate, followed by
d) administering a continuous IV infusion containing 2400 mg/m² 5-fluorouracil over 46 hours±1 hour followed by
e) administering an IV bolus containing 60 mg/m² (of BSA) [6R]-5,10-methylenetetrahydrofolate,
   wherein the location of the primary tumor of said patient has been determined to be left-sided or rectal, and wherein all steps a)-e) are repeated every 2 weeks until termination of the treatment, and wherein all steps a)-e) are repeated every 2 weeks for at least 16 weeks, until termination of the treatment.

Throughout the present application the treatment regimen according to the first aspect is referred to as the “ARFOX” protocol, and the treatment regimen according to the second aspect is referred to as the “ARFIRI” protocol.

The treatment based on the ARFOX or ARFIRI protocol may in principle be terminated “for any reason”, such as e.g. by a patient decision or a decision taken by the responsible medical person, i.a. due to disease progression or adverse events. Furthermore, the ARFOX or ARFIRI protocol may be interrupted by treatment holidays and the like. Finally the responsible medical person may decide on a fixed number of treatment cycles.

In particular embodiments of the first or second aspect, the primary tumor of said patient has been determined to be left-sided. In other embodiments of the first or second aspect, the primary tumor of said patient has been determined to be located in the distal one-third of the transverse colon, the descending colon or the sigmoid colon. In still further embodiments of the first or second aspect, the primary tumor of said patient has been determined to be rectal.

As mentioned above, several of the LCRC or RCA patients were also determined by genotype testing to be BRAF- or KRAS-mutation positive. In CRC patients the KRAS and BRAF mutation status is traditionally determined by tumor sample analysis. This requires surgery, and the subsequent analysis (extraction of genomic DNA from the tumor biopsy and analysis for mutations using dPCR) often takes weeks to complete. This creates problems in clinical situations which require urgent treatment based on the mutation status of the patient.

However, several studies the past 5-10 years have demonstrated that genotype testing by analysis of circulating, cell-free tumor DNA (ctDNA) in plasma or serum samples is becoming increasingly accurate and thus important as a non-invasive and fast alternative or supplement to tumor sample analysis. The method is also referred to as “Liquid Biopsy” analysis. Cell-free DNA (cfDNA) is fragmented DNA that is found in the non-cellular blood components. Among tumor patients, ctDNA is 150-200 base pair fragments that are released by tumor cells into the bloodstream and represents a small fraction of the total cfDNA. Importantly, ctDNA retains epigenetic characteristics and carries tumor-specific mutations that can be detected in peripheral blood (Bi 2020). Analysis of ctDNA in plasma is based on sequencing assays, see eg Finkle 2021.

It was thus reported (Mas 2019) that in a study involving four hundred and twenty-five enrolled mCRC patients, the paired tumor tissue and plasma samples of the patients showed an accuracy of 97.3% (95% CI: 95.2-98.6%) between the BRAF status in plasma and tissue for patients with available paired samples (n=405), and 98.5% (95% CI: 96.4-99.5%) for those with conclusive ctDNA (n=323). The absence of liver metastasis was the main factor associated with inconclusive ctDNA results. In patients with liver metastases, the accuracy was 98.6% (95% CI, 96.5-99.6%).

Similarly, another study (Bachet 2018) involving 329 patients with detectable ctDNA (at least one mutation or one methylated biomarker) showed an accuracy of 94.8% (95% CI, 91.9% to 97.0%) between the RAS mutation status in plasma and tissue. The absence of liver metastases also here was the main clinical factor associated with inconclusive ctDNA results.

Analysis of ctDNA (“liquid biopsy” analysis) is thus deemed an important tool for determining the relevant patient group according to the first or second aspect of the present invention.

Accordingly, in embodiments of the invention, [6R]-5,10-methylene-tetrahydrofolate is provided for use in a human patient in the treatment of left-sided colorectal or rectal cancer tumors, which treatment comprises performing steps a)-e) according to the first or second aspect of the invention, wherein the human patient has been found either by traditional tumor tissue analysis or in preferred embodiments by ctDNA (“liquid biopsy”) analysis to be either BRAF mutation-positive or KRAS mutation-positive or both BRAF mutation-positive and KRAS mutation-positive.

In other embodiments of the invention, [6R]-5,10-methylene-tetrahydrofolate is provided for use in a human patient in the treatment of left-sided colorectal or rectal cancer tumors, which treatment comprises performing steps a)-e) according to the first or second aspect of the invention, wherein the human patient is between 18-49 years old.

It has also surprisingly been discovered that administration of [6R]-MTHF and 5-FU according to the first or second aspect of the present invention over a treatment period of at least 16 weeks leads to a retardation or prevention of the progression of left-sided or rectal, colorectal cancer tumors in a human patient.

In a third aspect of the invention [6R]-5,10-methylene-tetrahydrofolate is therefore provided for use in the retardation or prevention of the progression in a human patient of left-sided or rectal colorectal cancer tumors, which comprises performing and repeating steps a) to e) according to the first or second aspect of the present invention, over a total treatment period of at least 16 weeks.

In a preferred embodiment of the third aspect, there is provided [6R]-5,10-methylene-tetrahydrofolate for use in the retardation or prevention of the progression of the progression in a human of left-sided or rectal colorectal cancer tumors, whereby steps a) to e) according to the first or second aspect of the present invention are performed and repeated over a total treatment period of at least 16 weeks, and whereby no statistically significant progression of said tumors is observed between 8 and 16 weeks after initiating treatment.

In a fourth aspect of the invention, there is provided a method for retardation or prevention of the progression in a human of left-sided or rectal colorectal cancer tumors, which method comprises performing and repeating steps a) to e) according to the first or second aspect of the present invention, over a total treatment period of at least 16 weeks.

In a preferred embodiment of the fourth aspect, there is provided a method for retardation or prevention of the progression in a human of left-sided or rectal colorectal cancer tumors, which method comprises performing and repeating steps a) to e) according to the first or second aspect of the present invention, over a total treatment period of at least 16 weeks, whereby no statistically significant progression of said tumors is observed between 8 and 16 weeks after initiating treatment.

During the performance of the follow-up study, ˜20% of the patients who had been diagnosed with left-sided CRC or rectal cancer and treated according to either the ARFOX protocol or ARFIRI protocol were additionally treated with bevacizumab during some time point in study.

In specific embodiments bevacizumab is administered to a human patient during the treatment period according to any of the aspects of the present invention. In further embodiments, bevacizumab is administered to a human patient at a dose of 5 mg/kg as an IV infusion every two weeks. In still further embodiments, bevacizumab administration is initiated 8 weeks after initiating treatment.

In some embodiments of any of the aspects of the invention, 5-fluorouracil (5-FU) is replaced by a fluorinated pyrimidine base such as capecitabine (Xeloda), ie. N4-pentyloxycarbonyl-5′-deoxy-5-fluorocytidine, tegafur, 5-fluoro-pyrimidinone, UFT, doxifluridine, 2′-deoxy-5 fluorouridine, 5′-deoxy-5-fluorouridine, 1-(2′-oxopropyl)-5-FU, and alkyl-carbonyl-5-FU, BOF-A2, ftorafur(TS-1), and S-1.

In preferred embodiments of any of the aspects of the invention, [6R]-5,10-methylenetetrahydrofolate ([6R]-MTHF) is employed as a solid form which is soluble in water, such as a lyophilizate or a salt, optionally stabilized by one or more suitable excipients and/or antioxidants such as citric acid or ascorbic acid or salt forms thereof.

In other preferred embodiments of any of the aspects of the invention the lyophilisate of 6R-MTHF is reconstituted in an aqueous media.

In other preferred embodiments of any of the aspects of the invention the lyophilisate of 6R-MTHF is prepared from 6R-MTHF hemisulfate salt.

In other preferred embodiments of any of the aspects of the invention the lyophilisate is prepared from 6R-MTHF hemisulfate salt and trisodium citrate dihydrate.

In preferred embodiments of any of the aspects of the invention, the intravenous bolus administration of steps (b), (c) and (e) occur over of a period of 10 minutes or less.

In preferred embodiments of any of the aspects of the invention, the intravenous bolus administration of steps (b), (c) and (e) occur over of a period of 5 minutes or less.

In preferred embodiments of any of the aspects of the invention, the intravenous bolus administration of steps (b), (c) and (e) occur over of a period of 3 minutes or less.

In preferred embodiments of any of the aspects of the invention, step (c) follows step (b) after a period of 30 minutes±5 minutes.

In preferred embodiments of any of the aspects of the invention, step (d) follows step (c) after a period of less than 60 minutes.

In preferred embodiments of any of the aspects of the invention, step (d) follows step (c) after a period of between 30 and 60 minutes.

### Examples

Arfolitixorin (formerly Modufolin®) is a folate-based biomodulator developed by applicant to improve the outcome of a range of antimetabolite treatments used within oncology. One of the therapeutic areas of specific interest included in the development program of Arfolitixorin is as biomodulator of 5-fluorouracil (5-FU) activity in standard treatment regime for advanced, metastatic CRC, such as Stage IV.

The drug substance in arfolitixorin is [6R]-5,10-MTHF described hereinabove, which is a stable formulation of the naturally occurring diastereoisomer of MTHF. As mentioned in the background section of the present application, [6R]-5,10-MTHF, shortened [6R]-MTHF, is also a metabolite of leucovorin (LV). Unlike LV, arfolitixorin does not need to undergo metabolism and may be directly involved in the formation of the FdUMP TS ternary complex discussed hereinabove.

Clinical Study ISO-CC-005 was an exploratory, Phase I/II multiple-centre study to be carried out in Stadium IV CRC patients. The study was designed to show clinical relevance for patients by characterizing the tolerability of four arfolitixorin dose levels (30, 60, 120, and 240 mg/m2) in six different standard clinical settings in the presence of fixed doses of 5-FU alone or in combination with either oxaliplatin, irinotecan, or oxaliplatin and bevacizumab.

The below Table shows the initial treatment protocol for the Chemotherapy Agents (Bevacizumab, Oxaliplatin, Irinotecan, and/or 5-FU) and of the Study Drug arfolitixorin (Modufolin®):

TABLE 1

| | | | | | | | 5-FU |
|---|---|---|---|---|---|---|---|
| | | Bevacizuma b | Oxaliplatin^{¶} | Irinotecan^{#} | | | At approx. |
| | | At aporox. | At approx. | At approx | 5-FU^{§}* | Modufolin ® | 35 minutes |
| | | −180 minutes | −60 minutes | −60 minutes | At 0 | At approx. | (46-hour |
| Treatment | | (infusion 30 | (infusion 15 | (fusion 30 | minute | 30 minutes | continuous |
| Arm | Cohort* | to 90 min) | to 120 min) | to 90 min) | (bolus) | (bolus) | infusion) ^{a} |
| Arm 1 | Cohort 1 | N/A | N/A | N/A | 500 mg/m² | 30 mg/m² | N/A |
| | Cohort 2 | N/A | N/A | N/A | 500 mg/m² | 60 mg/m² | N/A |
| | Cohort 8 | N/A | N/A | N/A | 500 mg/m² | 120 mg/m² | N/A |
| | Cohort 9 | N/A | N/A | N/A | 500 mg/m² | 245 mg/m² | N/A |
| Arm 2 | Cohort 4 | N/A | 85 mg/m² | N/A | 500 mg/m² | 30 mg/m² | N/A |
| | Cohort 5 | N/A | 85 mg/m² | N/A | 500 mg/m² | 60 mg/m² | N/A |
| Arm 3 | Cohort 6 | N/A | N/A | 180 mg/m² | 500 mg/m² | 30 mg/m² | N/A |
| | Cohort 7 | N/A | N/A | 180 mg/m² | 500 mg/m² | 60 mg/m² | N/A |
| Arm 4 | Cohort 12 | N/A | 85 mg/m² | N/A | 400 mg/m² | 60 mg/m² | 2 400 mg/m² |
| | Cohort 13 | N/A | 85 mg/m² | N/A | 400 mg/m² | 120 mg/m^{2 a} | 2 400 mg/m² |
| | Cohort 14 | N/A | 85 mg/m² | N/A | 400 mg/m² | 240 mg/m^{2 a} | 2 400 mg/m² |
| | Cohort 18a | N/A | 85 mg/m² | N/A | 400 mg/m² | SP2D ^{a} | 2 400 mg/m² |
| | Cohort 18b | | | | | | |
| Arm 5 | Cohort 15 | 5 mg/kg | 85 mg/m² | N/A | 400 mg/m² | 60 mg/m² | 2 400 mg/m² |
| | | | | | | or SP2D ^{a, b} | |
| | Cohort 16 | 5 mg/kg | 85 mg/m² | N/A | 400 mg/m² | 120 mg/m² | 2 400 mg/m² |
| | | | | | | or SP2D ^{a, b} | |
| | Cohort 17 | 5 mg/kg | 85 mg/m² | N/A | 400 mg/m² | 240 mg/m² | 2 400 mg/m² |
| | | | | | | or SP2D ^{a, b} | |
| Arm 6 | Cohort 19a | N/A | N/A | 180 mg/m² | 400 mg/m² | SP2D ^{a, b} | 2 400 mg/m² |
| | and 19b | | | | | | |

Abbreviation:
N/A: not applicable,
SP2D: selected phase 2 dose.
^{¶}The time-point window for Oxaliplatin administration will be expanded to allow infusion times of up to 120 minutes, if necessary
^{#}The time-point window for Irinotecan administration will be expanded to allow infusion times of up to 90 minutes, if necessary.
^{§}The administered bolus 5-FU dose should not surpass the maximum recommended daily dose of 1000 mg, regardless of the body surface area.
*Cohort #3, Cohort #10 and Cohort #11, originally included in earlier versions of this clinical study protocol, have been erased.
^{a} In Treatment Arm #4 (Cohorts #12, #13, #14, #18a, and #18b), Arm #5 (Cohort #15, #16, and #17, if applicable), and Arm #6 (Cohort #19a and #19b) the total Modufolin ® dose will be divided into two (2) i.v. bolus injections dispensed approximately 30 and 60 minutes after administration of 5-FU bolus injection (at 0 minute), respectively. The continuous 5-FU infusion will be paused for administration of the second Modufolin ® injection.
^{b} The SP2D is the Modufolin ® dose level in Treatment Arm #4 (MOFOX) assessed as the dose level with the most favourable profile for following investigation. The SP2D will be the highest Modufolin ® dose administered in Treatment Arm #5.

The tolerability of arfolitixorin was to be determined by the presence of Dose Limiting Toxicity (DLT) in each of the treatment arms and for each investigated arfolitixorin dose. For this, the safety of enrolled patients was closely monitored during the study with detailed rules for advancing to next dose cohort(s) or stopping the study.

The study was divided in the Main Study and the Follow-up Study. In the Main Study, patients received study treatment with arfolitixorin during eight weeks. Patients eligible for the Follow-up study could participate until reaching progress, but no longer than 18 months. The Main Study was divided into a dose-finding and a proof-of-concept part. The goal with the Dose-finding part of the Main Study was to establish the arfolitixorin dose level assessed as having the most favourable profile, i.e. the selected phase 2 dose (SP2D). The goal with the Proof-of-concept part of the Main Study was to acquire data on the safety and tolerability of arfolitixorin at the SP2D dose level in settings equivalent to the two well-established combination therapies FOLFOX (i.e. oxaliplatin/5-FU/LV) and FOLFIRI (i.e. irinotecan/5-FU/LV).

Enrolled patients, stadium IV CRC patients, were aware of the relatively poor prognosis of their disease. Those patients who could continue benefitting from treatment with a seemly effective therapy, were offered the possibility to continue study treatment by participating in the Follow-up study. In the Follow-up study patients continued to receive the same treatment as assigned during the Main Study period. However, the Investigator could complete the allocated treatment with other therapeutic agents of choice in alignment with standard of care in order to adapt treatment to the patient's specific needs and, in this way, provide optimal care.

Response was measured in short- and long-term assessments. During the Main Study phase, only short-term assessments of tumour response were explored by means of objective response rate (ORR) and early tumour shrinkage (ETS). These assessments were to be used as indicators of prognostic factor in ascertaining earlier non responders and to explore the correlation to other factors such as folate levels, tumour biomarkers, or expression levels of certain key genes.

As mentioned hereinabove, during a further analysis of the Follow-up study results applicant has now discovered that some of the tested combinations have proven surprisingly effective in patients diagnosed with left-sided colorectal (LCRC) or rectal cancer (RCA).

Some of the treated patients were further determined by genotype testing to be KRAS- and/or BRAF-mutation positive, i.e. cancer types which are notoriously difficult to treat.

The objectives in the Follow-up study were to:
To characterise all adverse events (AEs) and clinically significant abnormal laboratory test result changes regardless of attribution during the entire Follow-up Study period.
To evaluate tumour response and disease progression by means of ORR after every response evaluation since treatment allocation in the Main Study and as long patients continue in the Follow-up Study.
To evaluate tumour response and disease progression by means of PFS and TTP since treatment allocation in the Main Study and until the end of patient participation in the Follow-up Study.
To evaluate time-to-death since treatment allocation in the Main Study.
To evaluate the change in tumour biomarker (TK1) levels in blood after every four consecutive cycles of treatment with combination therapy in the subset of patients with available blood samples.

Correlation between tumour biomarkers in blood and treatment response by means of PFS and ORR as per RECIST 1.1 since baseline baseline in the Main Study was determined in in the subset of patients with available blood samples. Similarly, the correlation between tumour biomarkers in tumour tissue and treatment response by means of PFS and ORR as per RECIST 1.1 since baseline in the Main Study, was determined in the subset of patients with available tissue biopsy samples.

In the following, the main results of the Follow-up study will be discussed.

### RESULTS

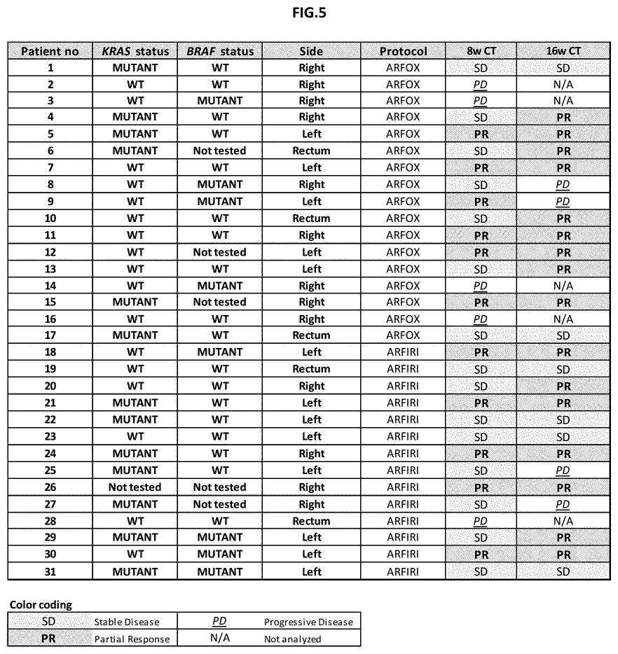

See FIG. 5 for a summarized overview of the 31 participating patients in the Follow-up study. In the following a more detailed, yet anonymized case narrative is given for each patient up to either 8 or 16 weeks treatment, depending on termination.

Patients had been randomized to either the ARFOX or ARFIRI treatment protocol before the follow-up study started, and remained on this protocol for the duration of the study unless otherwise indicated in the below narratives. Patients showing progressive disease (PD) by CT scanning at 8 weeks were not continued in the extension cohorts for another 8 weeks. Patients showing either partial response (PR) or stable disease (SD) at 8 weeks were as a rule continued for 8 more weeks, and assessed again by CT scanning at approximately 16 weeks (or later), i.e. at the 1st follow-up visit. Some patients were also continued for more than 16 weeks, but are not reported here.

The dates for individual events like e.g. genotype testing are shown in square brackets [nn].

### CASES

### Case #1

The patient is a 32-year-old white female randomized for treatment according to the ARFOX protocol (see above). Medical history findings at enrolment are hysterectomy and partial colectomy but no concomitant medication. The primary right sided tumour has been removed but no adjuvant therapy has been given. Genotype testing [2018-05-10] shows patient is KRAS mutant and BRAF wildtype, NRAS wildtype. Genotype testing [2019-04-24] shows patient is MSI stable. Baseline CT [2018-04-30] showed 1 target lesion in the liver (right lobe). At 8 week CT [2018-07-02] the sum of diameter of the target lesion(s) remained (stable disease) and patient was thereby eligible for participation in the follow-up study. At 1st follow-up visit [2018-08-27] CT the sum of diameter of the target lesion(s) decreased with 16% (stable disease). During study participation the following AEs were reported: dry eyes grade 1, fatigue grade 1 and neutrophil count decreased grade 2. The decreased neutrophil count was treated with filgrastim. During the follow-up study bevacizumab was added to the ARFOX treatment.

### Case #2

The patient is a 64-year-old white female randomized for treatment according to the ARFOX protocol (see above). The primary right sided tumour is still in place and no adjuvant therapy has been given. Genotype testing [2018-07-27] shows patient is KRAS, BRAF and NRAS wildtype. Baseline CT [2018-07-02] showed 2 target lesions in the liver parenchyma. At 8 week CT [2018-09-26] the sum of diameter of the target lesion(s) increased with 24% and additional lesions were discovered (progressive disease).

### Case #3

The patient is a 69-year-old white female randomized for treatment according to the ARFOX protocol (see above). No medical history findings and no concomitant medication at enrolment. The primary right sided tumour is still in place and no adjuvant therapy has been given. Genotype testing [2018-09-13] shows patient is KRAS wildtype, BRAF mutant, NRAS wildtype. Genotype testing [2019-02-14] shows patient is MMR stable. Baseline CT [2018-07-04] showed 1 target lesion in the liver (segment IV). At 8 week CT [2018-09-21] the sum of diameter of the target lesion(s) increased with 22% and additional lesions were discovered (progressive disease). No AEs nor concomitant medication reported during study participation.

### Case #4

A 85-year-old white female randomized for treatment according to the ARFOX protocol (see above). The primary right sided tumour has been removed and adjuvant therapy with CAPECITABINE has been given. Genotype testing [2018-09-13] shows patient is KRAS mutant and BRAF and NRAS wildtype. Baseline CT [2018-08-01] showed 1 target lesion in the left lower lobe of the lung. At 8 week CT [2018-10-08] the sum of diameter of the target lesion(s) decreased with 10% (stable disease) and patient consented to participation in the follow-up study. At 1st follow-up visit [2018-11-26] CT the sum of diameter of the target lesion(s) decreased with additional 30% (partial response). During the follow-up study bevacizumab was added to the ARFOX treatment.

### Case #5

The patient is a 69-year-old white female randomized for treatment according to the ARFOX protocol (see above). The primary left sided tumour has been removed but no adjuvant therapy has been given. Genotype testing [2018-09-13] shows patient is KRAS mutant and BRAF and NRAS wildtype. Baseline MRI [2018-08-09] showed 1 target lesion in the liver parenchyma. At 8 week CT [2018-11-13] the sum of diameter of the target lesion(s) decreased with 33% (partial responses) and patient consented to participation in the follow-up study. At 1st follow-up visit [2019-03-18] CT the sum of diameter of the target lesion(s) decreased with additional 25% (partial response).

### Case #6

The patient is a 71-year-old white male randomized for treatment according to the ARFOX protocol (see above). The primary rectal tumour is still in place and no adjuvant therapy has been given. Genotype testing [2018-08-24] shows patient is KRAS mutant and MLH1, PMS2 and MSH2 stable. Baseline CT [2018-09-14] showed 1 target lesion in the segment 6/7 of the liver. At 8 week CT [2018-11-05] the sum of diameter of the target lesion(s) decreased with 17% (stable disease) and patient consented to participation in the follow-up study. At 1st follow-up visit [2019-02-15] CT the sum of diameter of the target lesion(s) decrease with additional 25% (partial response).

### Case #7

The patient is a 75-year-old white male randomized for treatment according to the ARFOX protocol (see above). Medical history findings at enrolment are asthma that is treated with Symbicort, and sigmoidectomy. No other concomitant medication. The primary left sided tumour has been removed but no adjuvant therapy has been given. Genotype testing [2018-11-06] shows patient is KRAS, BRAF and NRAS wildtype. Baseline CT [2018-10-12] showed 3 target lesions; 2 in the lung (left and right lower lobe) and 1 in the liver (left lobe). At 8 week CT [2018-12-15] the sum of diameter of the target lesion(s) decreased with 64% (partial responses) and patient consented to participation in the follow-up study. At 1st follow-up visit [2019-02-23] CT the sum of diameter of the target lesion(s) increased with 10% (partial response). During study participation the following AEs were reported: nausea, weight loss and thrombocytopenia, all grade 1. No other concomitant medication during main study in addition to the Symbicort patient had at enrollment in the study. During the follow-up study bevacizumab was added to the ARFOX treatment.

### Case #8

The patient is a 62-year-old white male randomized for treatment according to the ARFOX protocol (see above). The only medical history finding at enrolment is right colectomy and no concomitant medication. The primary right sided tumour has been removed but no adjuvant therapy has been given. Genotype testing [2018-10-16] shows patient is KRAS wildtype, BRAF mutant, NRAS wildtype. Genotype testing [2018-10-24] shows patient is MSI negative. Baseline CT [2018-10-17] showed 5 target lesions in the liver, lung and tumour deposit. At 8 week CT [2018-12-19] the sum of diameter of the target lesion(s) decreased with 6% (stable disease) and patient consented to participation in the follow-up study. At 1st follow-up visit [2019-03-13] CT the sum of diameter of the target lesion(s) increased with 1% but additional lesions were discovered in the lung (progressive disease). During main study SAE pulmonary embolism [2019-12-18] was reported. The following non-serious AEs were reported during main or follow-up: nausea, fatigue, anemia and weight loss. Bevacizumab treatment was initiated during the follow-up study.

### Case #9

The patient is a 61-year-old white female randomized for treatment according to the ARFOX protocol (see above). Medical history findings at enrolment are hyperuricemia and hypertension that are both treated. The primary left sided tumour has been removed but no adjuvant therapy has been given. Genotype testing [2018-10-17] shows patient is KRAS wildtype, BRAF mutant, NRAS wildtype and MSI negative. Baseline CT [2018-10-25] showed 3 target lesions in the liver (segment IV, VI and VII). At 8 week CT [2018-12-27] the sum of diameter of the target lesion(s) decreased with 37% (partial response) and patient consented to participation in the follow-up study. At 1st follow-up visit [2019-03-06] CT the sum of diameter of the target lesion(s) decrease with 15% but additional lesions were discovered (progressive disease). The only AE reported during patient's participation in the study was fatigue grade 1. Bevacizumab treatment was initiated during the follow-up study in addition to the concomitant medication patient had at enrollment in the study.

### Case #10

The patient is a 48-year-old white male randomized for treatment according to the ARFOX protocol (see above). The primary rectal tumour is still in place and no adjuvant therapy has been given. Genotype testing [2018-11-26] shows patient is KRAS, BRAF and NRAS wildtype. Baseline CT+MRI [2018-11-12] showed 3 target lesions; in the liver lobe, in the pericolonic lymph nodes (lymph nodes) and in left pelvis. At 8 week CT [2019-02-09] the sum of diameter of the target lesion(s) decreased with 14% (stable disease) and patient was thereby eligible for participation in the follow-up study. At 1st follow-up visit [2019-05-02] CT the sum of diameter of the target lesion(s) decreases with additional 59% (partial response).

### Case #11

The patient is a 67-year-old white female randomized for treatment according to the ARFOX protocol (see above). Medical history findings at enrolment are an ostomy surgery in the past and an ongoing candida infection, but no concomitant medication. The primary right sided tumour is still in place and no adjuvant therapy has been given. Genotype testing [2018-12-12] shows patient is KRAS and BRAF wildtype, NRAS mutant. Baseline CT [2018-12-16] showed 2 target lesions in the liver (right lobe dorsal lateral). At 8 week CT [2019-02-26] the sum of diameter of the target lesion(s) decreased with 40% (partial response) and patient consented to participation in the follow-up study. At 1st follow-up visit [2019-04-25] CT the sum of diameter of the target lesion(s) decreased with an additional 29% (partial response). During study participation the following AEs were reported: dry skin grade 1, treated with Canoderm, ileostomy infection grade 2, treated with antibiotics, insomnia grade 1 and loss of appetite grade 2. Patient also had a number of AEs related to bone marrow toxicity (neutropenia, leukopenia) with grade ranging from 1-3—treated accordingly with Zarzio, and a couple of occasions of nausea grade 1 despite a number of prophylactic drugs given. During participation patient also developed neuropathy grade 1. During study participation, patient also received thrombosis prophylaxis and constipation prophylaxis. Patient's candida infection [MEI] was treated with fluconazole and nystimex.

### Case #12

The patient is a 69-year-old white male randomized for treatment according to the ARFOX protocol (see above). The primary left sided tumour has been removed but no adjuvant therapy has been given. Genotype testing [2018-12-28] shows patient is KRAS and NRAS wildtype. Baseline CT [2018-12-03] showed 3 target lesions; in the part IV of the liver, in left adrenal gland and in lung nodules. At 8 week CT [2019-02-27] the sum of diameter of the target lesion(s) decreased with 36% (partial responses) and patient consented to participation in the follow-up study. At 1st follow-up visit [2019-04-24] CT the sum of diameter of the target lesion(s) decrease with additional 33% (partial response).

### Case #13

The patient is a 34-year-old white female randomized for treatment according to the ARFOX protocol (see above). No medical history findings and no concomitant medication at enrolment. The primary left sided tumour has been removed but no adjuvant therapy has been given. Genotype testing [2019-01-31] shows patient is KRAS, BRAF and NRAS wildtype. Baseline MRI [2019-01-02] showed 2 target lesions in the liver (left and right lobe). At 8 week CT [2019-02-28] the sum of diameter of the target lesion(s) decreased with 18% (stable disease) and patient consented to participation in the follow-up study. At 1st follow-up visit [2019-05-13] CT the sum of diameter of the target lesion(s) decreased with an additional 60% (partial response). No AEs nor concomitant medication reported during main study, but panitimumab was added to the ARFOX treatment during the follow-up study.

### Case #14

The patient is a 53-year-old white male randomized for treatment according to the ARFOX protocol (see above). Medical history finding at enrolment is depression, which is treated with Sobril. Other concomitant medication at enrolment is treatment of pain, heartburn, rhinit as well as constipation and thrombosis prophylaxes. The primary right sided tumour is still in place and no adjuvant therapy has been given. Genotype testing [2019-01-09] shows patient is KRAS wildtype, BRAF mutant, NRAS wildtype and MSS negative. Baseline CT [2019-01-02] showed 3 target lesions in the liver (segment IV and VII) and lymph node. At 8 week CT [2019-03-27] the sum of diameter of the target lesion(s) increased with 29% and additional lesions were discovered (progressive disease).

### Case #15

The patient is a 68-year-old white male randomized for treatment according to the ARFOX protocol (see above). The primary right sided tumour has been removed but no adjuvant therapy has been given. Genotype testing [2019-01-31] shows patient is KRAS mutant and MSS negative. Baseline CT [2019-02-21] showed 2 target lesions; in segment 6/7 and segment 8 of the liver. At 8 week CT [2019-04-15] the sum of diameter of the target lesion(s) decreased with 53% (partial responses) and patient was thereby eligible for participation in the follow-up study. At 1st follow-up visit [2019-06-12] CT the sum of diameter of the target lesion(s) decrease with additional 30% (partial response).

### Case #16

The patient is a 70-year-old white male randomized for treatment according to the ARFOX protocol (see above). The primary right sided tumour has been removed but no adjuvant therapy has been given. Genotype testing [2018-09-26] shows patient is MSI stable (microsatellite instability absent). Genotype testing [2020-02-04] shows patient is KRAS, BRAF and NRAS wildtype. Baseline CT [2019-03-06] showed 1 target lesion in the liver. At 8 week CT [2019-06-07] the sum of diameter of the target lesion(s) remained but additional lesions were discovered in the lung (progressive disease).

### Case #17

The patient is a 66-year-old white female randomized for treatment according to the ARFOX protocol (see above). The primary rectal tumour is still in place and no adjuvant therapy has been given. Genotype testing [2019-01-08] shows patient is KRAS mutant and BRAF and NRAS wildtype. Baseline CT [2019-03-07] showed 2 target lesions; in the left lower lung lobe and in the right upper lung lobe. At 8 week CT [2019-05-17] the sum of diameter of the target lesion(s) decreased with 12% (stable disease) and patient consented to participation in the follow-up study. At 1st follow-up visit [2019-07-29] CT the sum of diameter of the target lesion(s) remained (stable disease).

### Case #18

The patient is a 68-year-old white male randomized for treatment according to the ARFIRI protocol (see above). Medical history findings at enrolment are depression, gastroesophageal reflux and insomnia which are treated accordingly. Other medical history findings are untreated atrial fibrillation grade 2 and back pain. Patient also has liver surgery and hemicolectomy reported as medical history. The primary left sided tumour has been removed and adjuvant therapy with FOLFOX [EOT 2017-01-05] has been given. Genotype testing shows patient is KRAS wildtype, BRAF mutant, NRAS wildtype. Baseline MRI [2018-03-06] showed 1 target lesion in the liver (right dorsal). At 8 week MRI [2018-05-28] the sum of diameter of the target lesion(s) decreased with 30% (partial response) and patient consented to participation in the follow-up study. At 1st follow-up visit [2018-08-15] MRI the sum of diameter of the target lesion(s) decreased with an additional 40% (partial response). During the study, the patient had severe problems with GI related toxicity such as nausea and vomiting. Patient was allowed to try the following 5-FU bolus regimen (irinotecan 180 mg/m2 on day 1, bolus 5-FU 500 mg/m2 and arfolitixorin 60 mg/m2 on day 1 and 2) during the follow-up study without any significant change of toxicity. Patient also reported a number of occasions of fatigue grade 1-2 during study participation and initially one episode of paroxysmal atrial fibrillation grade 3 and a month later atrial fibrillation grade 3—both reported to be related to the study drug by the investigator. During follow-up study the patient twice receives radiotherapy due to AE costal pain.

### Case #19

The patient is a 65-year-old white female randomized for treatment according to the ARFIRI protocol (see above). The primary rectal tumour is still in place and adjuvant therapy with CAPECITABINE-OXALIPLATIN [EOT 2016-01-29] has been given. Genotype testing [2018-03-22] shows patient is KRAS, BRAF and NRAS wildtype. Baseline CT [2018-03-27] showed 1 target lesion in the pelvis (cervix, near rectal stump). At 8 week CT [2018-06-22] the sum of diameter of the target lesion(s) remained (stable disease) and patient was thereby eligible for participation in the follow-up study. At 1st follow-up visit [2018-09-03] CT the sum of diameter of the target lesion(s) remained (stable disease). Patient terminated study due to AE ‘Thromboembolic event’ [2018-09-03] after 1st follow-up visit. During the follow-up study bevacizumab was added to the ARFIRI treatment.

### Case #20

The patient is a 74-year-old white male randomized for treatment according to the ARFIRI protocol (see above). Medical history findings at enrolment are hypertension that is treated accordingly, hyperlipidemia and a right hemicolectomy. Patient is treated with Salospir and Placol as cardiovascular prevention. The primary right sided tumour has been removed and adjuvant therapy with CAPECITABINE-OXALIPLATIN [EOT 2017-UNK-UNK] has been given. Genotype testing [2018-05-04] shows patient is KRAS, BRAF and NRAS wildtype. Baseline CT [2018-05-14] showed 5 target lesions in the lung (right and left lobe), abdomen and abdominal aorta. At 8 week CT [2018-07-10] the sum of diameter of the target lesion(s) decreased with 27% (stable disease) and patient was thereby eligible for participation in the follow-up study. At 1st follow-up visit [2018-08-31] CT the sum of diameter of the target lesion(s) decreased with an additional 10% (partial response). Patient terminated study due to PI decision due to maximum clinical benefit [2018-10-05]. The only AE reported was a diarrhea grade 1 during follow-up study and during the follow-up study bevacizumab was added to the ARFIRI treatment. No other concomitant medication in addition to the concomitant medication patient had at enrollment in the study.

### Case #21

The patient is a 67-year-old white male randomized for treatment according to the ARFIRI protocol (see above). The primary left sided tumour is still in place and no adjuvant therapy has been given. Genotype testing [2018-05-22] shows patient is KRAS mutant, BRAF wildtype and MLH1, PMS2, MSH2 and MSH6 stable. Baseline CT [2018-04-16] showed 3 target lesions in the liver; 1 in left lobe apical and 2 in right lobe. At 8 week CT [2018-07-09] the sum of diameter of the target lesion(s) decreased with 42% (partial responses) and patient consented to participation in the follow-up study. At 1st follow-up visit [2018-10-17] CT the sum of diameter of the target lesion(s) decreased with additional 43% (partial response).

### Case #22

The patient is a 58-year-old white male randomized for treatment according to the ARFIRI protocol (see above). The primary left sided tumour has been removed but no adjuvant therapy has been given. Genotype testing [2017-11-28] shows patient is KRAS mutant and BRAF and NRAS wildtype. Baseline CT [2018-07-23] showed 2 target lesions in the lung; right lower lobe and lymphnode. At 8 week CT [2018-09-26] the sum of diameter of the target lesion(s) remained (stable disease) and patient consented to participation in the follow-up study. At 1st follow-up visit [2018-12-13] CT the sum of diameter of the target lesion(s) remained (stable disease). During the follow-up study bevacizumab was added to the ARFIRI treatment.

### Case #23

The patient is a 68-year-old white male randomized for treatment according to the ARFIRI protocol (see above). Medical history findings at enrolment are hypertension and restless legs that are treated accordingly. At enrolment, a Peripherally inserted central catheterization is done. No other concomitant medication. The primary left sided tumour has been removed but no adjuvant therapy has been given. Genotype testing [2018-08-10] shows patient is KRAS wildtype, BRAF wildtype and MLH1, PMS2, MSH2 and MSH6 stable. Baseline MRI [2018-07-06] showed 2 target lesions in the liver (segment 1 and 7). At 8 week MRI [2018-10-01] the sum of diameter of the target lesion(s) decreased with 13% (stable disease) and patient consented to participation in the follow-up study. At 1st follow-up visit [2019-01-10] MRI the sum of diameter of the target lesion(s) decrease with an additional 20% (stable disease). During the first 8 weeks of study participation the following AEs were reported: hypotension grade 2, insomnia grade 1 and worsening of restless legs (grade 1) that was reported as medical history. Beside treatment of the AEs reported, patient also received constipation prophylaxis, thrombosis prophylaxis and nausea prophylaxis.

### Case #24

The patient is a 58-year-old white female randomized for treatment according to the ARFIRI protocol (see above). Medical history findings at enrolment are depression and pain that are treated accordingly. Other medical history findings are twisted ovarian cyst, struma [goitre], anorexia, rash, fatigue and dry mouth. Medication for rash is prescribed at enrolment. The primary right sided tumour has been removed but no adjuvant therapy has been given. Genotype testing [2018-07-31] shows patient is KRAS mutant and BRAF wildtype, NRAS wildtype. Baseline CT [2018-08-22] showed 2 target lesions in retroperitoneal lymph node. At 8 week CT [2018-10-22] the sum of diameter of the target lesion(s) decreased with 47% (partial response) and patient consented to participation in the follow-up study. At 1st follow-up visit [2018-12-17] CT the sum of diameter of the target lesion(s) decreased with an additional 32% (partial response). Dizziness grade 1 and nausea grade 1 are reported at almost every treatment cycle, and nausea is treated with both oral an i.v. nausea prophylaxis. Patient also reports several episodes of epistaxis grade 1. Dry skin grade 1 is reported a couple of times and so is pain grade 1, lasting for several weeks at a time. During follow-up study oral mucositis lasting for more than a month is reported, treated with chamomile flower tea.

### Case #25

The patient is a 65-year-old white male randomized for treatment according to the ARFIRI protocol (see above). The primary left sided tumour has been removed but no adjuvant therapy has been given. Genotype testing [2017-10-12] shows patient is KRAS mutant and BRAF wildtype. Genotype testing [2017-10-13] shows patient is MSI negative. Baseline CT [2018-08-23] showed 1 target lesion in segment VI of the liver. At 8 week CT [2018-10-31] the sum of diameter of the target lesion(s) remained (stable disease) and patient consented to participation in the follow-up study. At 1st follow-up visit [2019-01-09] CT the sum of diameter of the target lesion(s) remained but additional lesions were discovered (progressive disease).

### Case #26

The patient is a 63-year-old white male randomized for treatment according to the ARFIRI protocol (see above). The primary right sided tumour has been removed but no adjuvant therapy has been given. No genotype testing performed. Baseline CT [2018-10-29] showed 2 target lesions; left ventral (gland) and left aorta (gland). At 8 week CT [2018-12-18] the sum of diameter of the target lesion(s) decreased with 54% (partial response) and patient consented to participation in the follow-up study. At 1st follow-up visit [2019-02-20] CT the sum of diameter of the target lesion(s) decreased with additional 8% (partial response).

### Case #27

The patient is a 45-year-old white male randomized for treatment according to the ARFIRI protocol (see above). The primary right sided tumour has been removed and adjuvant therapy with FLOX [EOT 2017-06-29] has been given. Genotype testing [2018-08-06] shows patient is KRAS mutant and MLH1, PMS2, MSH2 and MSH6 stable. Baseline CT [2018-12-07] showed 1 target lesion the right lower lobe of the lung, ventral. At 8 week CT [2019-02-11] the sum of diameter of the target lesion(s) remained (stable disease) and patient consented to participation in the follow-up study. At 1st follow-up visit [2019-05-20] CT the sum of diameter of the target lesion(s) increased with 50% (progressive disease).

### Case #28

The patient is a 46-year-old Asian female randomized for treatment according to the ARFIRI protocol (see above). The primary rectal tumour is still in place and no adjuvant therapy has been given. Genotype testing [2018-11-16] shows patient is KRAS and BRAF wildtype and NRAS mutant. Baseline CT [2018-12-27] showed 2 target lesions in the liver. At 8 week CT [2019-04-08] the sum of diameter of the target lesion(s) increased with 80% (progressive disease).

### Case #29

The patient is a 72-year-old white female randomized for treatment according to the ARFIRI protocol (see above). Medical history finding at enrolment is (mild) hypertension treated with hydrochlorothiazide. No other medical history finding nor concomitant medication. The primary left sided tumour has been removed but no adjuvant therapy has been given. Genotype testing [2019-03-01] shows patient is KRAS and BRAF mutant, NRAS mutant. Genotype testing [2019-12-12] shows patient is MSI stable (microsatellite instability absent). Baseline CT [2019-03-19] showed 1 target lesion in the lung (left upper lobe). At 8 week CT [2019-05-11] the sum of diameter of the target lesion(s) remained (stable disease) and patient consented to participation in the follow-up study. At 1st follow-up visit [2019-07-19] CT the sum of diameter of the target lesion(s) decreased with 44% (partial response). No AEs and no other concomitant medication in addition to the concomitant medication patient had at enrollment in the study.

### Case #30

The patient is a 68-year-old white female randomized for treatment according to the ARFIRI protocol (see above). Medical history findings at enrolment are hypercholesterolemia, paroxysmal atrial tachycardia and chronic respiratory failure which are treated accordingly. The primary left sided tumour has been removed but no adjuvant therapy has been given. Genotype testing [2019-04-11] shows patient is BRAF mutant and MSI stable (microsatellite instability absent). Genotype testing [2019-04-16] shows patient is KRAS wildtype. Genotype testing [2019-04-19] shows patient is NRAS wildtype. Baseline CT [2019-04-03] showed 3 target lesions in the liver (right lobe) and lung (right lobe). At 8 week CT [2019-06-04] the sum of diameter of the target lesion(s) decreased with 54% (partial response) and patient consented to participation in the follow-up study. At 1st follow-up visit [2019-08-02] CT the sum of diameter of the target lesion(s) decreased with an additional 39% (partial response). Two AEs are reported during follow-up study; diarrhea and fatigue grade 2. No other concomitant medication in addition to the concomitant medication patient had at enrollment in the study.

### Case #31

The patient is a 52-year-old white female randomized for treatment according to the ARFIRI protocol (see above). No medical history findings and no concomitant medication at enrolment. The primary left sided tumour has been removed but no adjuvant therapy has been given. Genotype testing [2019-04-25] shows patient is KRAS and BRAF mutant, NRAS wildtype. Genotype testing [2019-04-19] shows patient is MSI stable (microsatellite instability absent). Baseline CT [2019-03-13] showed 2 target lesions in the lung (left and right lower lobe). At 8 week CT+MRI [2019-06-06] the sum of diameter of the target lesion(s) decreased with 15% (stable disease) and patient consented to participation in the follow-up study. At 1st follow-up visit [2019-07-29] CT the sum of diameter of the target lesion(s) increased with 3% (stable disease). Patient terminated study due to patient's request [2019-08-06]. No AEs nor concomitant medication reported during study participation.

### REFERENCES

1. F Caputo et al. BRAF-Mutated Colorectal Cancer: Clinical and Molecular Insights. Int. J. Mol. Sci. 2019, 20, 5369
2. F Bray, et al. Global cancer statistics 2018: GLOBOCAN estimates of incidence and mortality worldwide for 36 cancers in 185 countries. CA Cancer J. Clin. 2018, 68, 394-424.
3. American Cancer Society. Cancer Facts & Figures; American Cancer Society: Atlanta, Ga., USA, 2016.
4. C S Fuchs et al. Randomized, controlled trial of irinotecan plus infusional, bolus, or oral fluoropyrimidines in first-line treatment of metastatic colorectal cancer: Results from the BICC-C Study. J. Clin. Oncol. 2007, 25, 4779-4786.
5. J Tabernero et al. Unmet Medical Need in Patients with Metastatic Colorectal Cancer with BRAF V600E Mutations: A Review. EMJ Oncol. 2020; 8[Suppl 3]: 2-14.
6. B Baran et al. Difference Between Left-Sided and Right-Sided Colorectal Cancer: A Focused Review of Literature. Gastroenterol Res. 2018; 11(4):264-273
7. CM Ribic et al. Tumor Microsatellite-Instability Status as a Predictor of Benefit from Fluorouracil-Based Adjuvant Chemotherapy for Colon Cancer. N Engl J Med. 2003; 349(3):247-257
8. BM Wolpin et al. Systematic treatment of colorectal cancer. Gastroenterol. 2008; 134(5):1296 1310
9. H Brenner et al. Colorectal cancer. Lancet. 2014; 383(9927):1490-1502.
10. Peng et al. Right- and left-sided stage III colon cancers present different prognostic outcomes of oxaliplatin-based adjuvant chemotherapy after curative resection. Cancer Management and Research 2018:10
11. FA Sinicrope et al. Prognostic Impact of Deficient DNA Mismatch Repair in Patients With Stage III Colon Cancer From a Randomized Trial of FOLFOX-Based Adjuvant Chemotherapy. J Clin Oncol. 2013; 31(29):3664-3672.
12. C Cremolini et al. When to Use Triplet Chemotherapy as First-Line Treatment in Metastatic Colorectal Cancer. Clinical Advances in Hematology & Oncology August 2019-Volume 17, Issue 8
13. C Cremolini et al. Individual Patient Data Meta-Analysis of FOLFOXIRI Plus Bevacizumab Versus Doublets Plus Bevacizumab as Initial Therapy of Unresectable Metastatic Colorectal Cancer. Journal of Clinical Oncology 38(28) August 2020
14. M SIDERIS et al. BRAF V600E Mutation in Colorectal Cancer Is Associated with Right-sided Tumours and Iron Deficiency Anaemia. ANTICANCER RESEARCH 35: 2345-2350 (2015)
15. P Ross Right versus left-sided colon cancer: Is it time to consider these as different diseases? AIMS Medical Science, 5(3): 303-315 (2018)
16. D Hanna, How We Treat Left-Sided vs Right-Sided Colon Cancer. Clinical Advances in Hematology & Oncology Volume 18, Issue 5 May 2020
17. H Taniguchi et al. Tumor Location Is Associated With the Prevalence of Braf And Pik3ca Mutations in Patients with Wild-Type Ras Colorectal Cancer: A Prospective Multi-Center Cohort Study in Japan. Translational Oncology 13 (2020) 100786
18. Li et al. Gastroenterology Report, 8(3), 2020, 192-205
19. Greystoke et al. How Many Diseases Are Colorectal Cancer? Gastroenterology Research and Practice, Vol. 2012, p. 1-12
20. Gomez et al. Anatomical distribution of colorectal cancer over a 10 year period in a district general hospital: is there a true “rightward shift?” Postgrad Med J 2004; 80:667-669
21. Grassadonia et al. Impact of primary tumor location in patients with RAS wild-type metastatic colon cancer treated with first-line chemotherapy plus anti-EGFR or anti-VEGF monoclonal antibodies: a retrospective multicenter study. Journal of Cancer 2019, Vol. 10, 5926
22. Recio-Boiles A, et al. Rectal Cancer. [Updated 2020 Dec. 17]. In: StatPearls [Internet]. Treasure Island (FL): StatPearls Publ. 2021. Available from: https://www.ncbi.nlm.nih.gov/books/NBK493202/
23. Sun, Dc., et al. KRAS mutation and primary tumor location do not affect efficacy of bevacizumab-containing chemotherapy in stagae IV colorectal cancer patients. Sci Rep 7, 14368 (2017).
24. Koumarianou A et al., Implications of KRAS status in first line chemotherapy with bevacizumab in advanced colorectal cancer: A phase IV study of Hellenic Oncology Research Group (HORG). Journal of Clinical Oncology 2018 36:15 suppl, e15521-e15521
25. Sanford N et al., Early-onset colorectal cancer: more than one side to the story. COLORECTAL CANCER VOL. 9, NO. 3 (2020)
26. Burge M et al., First-line therapy for metastatic colorectal cancer: Current perspectives and future directions. Asia-Pac J Clin Oncol. 2019; 15(Suppl. 1):3-14.
27. Giuliani et al., Int J Colorectal Dis. 2018 November; 33(11): 1505-1516 (2018)
28. A Venook et al., Impact of primary(1°) tumor location on overall survival (OS) and progression-free survival (PFS) in patients (pts) with metastatic colorectal cancer (mCRC): Analysis of CALGB/SWOG 80405 (Alliance). Journal of Clinical Oncology 34, no. 15_suppl (May 20, 2016) 3504-3504.
29. V Gausman et al. Risk Factors Associated With Early-Onset Colorectal Cancer. Clin Gastroenterol Hepatol. 2020 November; 18(12):2752-2759.e2. doi: 10.1016/j.cgh.2019.10.009.
30. G Mauri et al. Early-onset colorectal cancer in young individuals. Molecular Oncology 13 (2019) 109-13.
31. DJ Sargent et al. A pooled analysis of adjuvant chemotherapy for resected colon cancer in elderly patients. N Engl J Med 345 (15): 1091-7, 2001
32. F Loupakis et al. Impact of primary tumour location on efficacy of bevacizumab plus chemotherapy in metastatic colorectal cancer. BJC (2018) 119:1451-1455
33. Van Cutsem et al. Fluorouracil, leucovorin, and irinotecan plus cetuximab treatment and RAS mutations in colorectal cancer. J. Clin Oncol. (2015) 33:692-700
34. KM Tveit et al. Phase III trial of cetuximab with continuous or intermittent fluorouracil, leucovorin, and oxaliplatin (Nordic FLOX) versus FLOX alone in first-line treatment of metastatic colorectal cancer: the NORDIC-VII study J. Clin Oncol. (2012) 30(15):1755-1762)

What is claimed is:

1. A method of treating a human patient diagnosed with a solid tumor colorectal cancer or metastatic colorectal cancer comprising the following steps:
a) selecting a human patient with a left-sided colorectal or metastatic colorectal solid tumor,
b) administering to said patient a continuous intravenous (IV) infusion of a pharmaceutical composition comprising either
i. 85 mg/m² (of BSA) oxaliplatin, or
ii. 180 mg/m² (of BSA) irinotecan,
followed by
c) administering to said patient an IV bolus of a pharmaceutical composition comprising 400 mg/m² (of BSA) 5-fluorouracil (5-FU), followed by
d) administering to said patient an IV bolus of a pharmaceutical composition comprising 60 mg/m2 [6R]-5,10-methylene-tetrahydrofolate (6R-MTHF), followed by
e) administering to said patient a continuous IV infusion of a pharmaceutical composition comprising 2400 mg/m2 5-FU over 46 hours±1 hour followed by
f) administering to said patient an IV bolus of a pharmaceutical composition comprising 60 mg/m2 (of BSA) 6R-MTHF.

2. The method of claim 1, wherein said left-sided colorectal or metastatic colorectal solid tumor is located in the descending and sigmoid colon, or distal one third of the transverse colon.

3. The method of claim 1, wherein said left-sided colorectal or metastatic colorectal solid tumor is located in the rectum.

4. The method of claim 1, wherein steps b)-f) are repeated every 2 weeks for a total treatment period of at least 16 weeks.

5. The method of claim 1, wherein steps b)-f) are repeated every 2 weeks until termination of the treatment.

6. The method of claim 1, further comprising administering to said patient during the treatment period a pharmaceutical composition comprising bevacizumab.

7. The method of claim 5, wherein the pharmaceutical composition comprising 5 mg/kg of the pharmaceutical composition comprising bevacizumab is administered as an IV infusion every two weeks.

8. The method of claim 6, wherein bevacizumab administration begins 8 weeks after initiating step b) of the treatment.

9. The method of claim 6, wherein bevacizumab administration begins prior to initiating step b) of the treatment.

10. The method of claim 1, wherein the 6R-MTHF is reconstituted from a lyophilisate prior to administration.

11. The method of claim 11, wherein the lyophilisate is reconstituted in aqueous media.

12. The method of claim 11, wherein the lyophilisate of 6R-MTHF is prepared from 6R-MTHF hemisulfate salt.

13. The method of claim 13, wherein the lyophilisate is prepared from 6R-MTHF hemisulfate salt and trisodium citrate dihydrate.

14. The method of claim 1, wherein the pharmaceutical composition comprising 6R-MTHF further comprises citric or ascorbic acid or salts thereof.

15. The method of claim 1, wherein the 6R-MTHF has a diastereomeric purity of >98% d.e.

16. The method of claim 2, wherein said method retards or inhibits progression of said solid tumors.

17. The method of claim 16, wherein said method produces no statistically significant progression of said solid tumors up to at least 16 weeks after initiating treatment.

18. The method of claim 1, wherein the intravenous bolus administration of steps (c), (d) and (f) occur over of a period of 10 minutes or less.

19. The method of claim 1, wherein the intravenous bolus administration of step (c), (d) or (f) occur over a period of 5 minutes or less.

20. The method of claim 1, wherein the intravenous bolus administration of step (c), (d) or (f) occur over a period of 3 minutes or less.

21. The method of claim 1, wherein step (d) follows step (c) after a period of 30 minutes±5 minutes.

22. The method of claim 1, wherein step (e) follows step (d) after a period of less than 60 minutes.

23. The method of claim 1, wherein step (e) follows step (d) after a period of between 30 and 60 minutes.

24. The method of claim 1, wherein said human patient is either BRAF mutation positive or KRAS mutation-positive or both BRAF mutation positive and KRAS mutation positive.
